(19) United States
(12) Patent Application Publication
(10) Pub. No.: US 2004/0200477 A1
(43) Pub. Date: Oct. 14, 2004

(54) Portable assembly for emergency ventilation

(76) Inventors: Christian Bleys, Livry s/Seine (FR); Noureddine Kissi, Meudon Ia Foret (FR)

Correspondence Address: Linda K. Russell, Air Liquide, Suite 1800, 2700 Post Oak Blvd., Houston, TX, 77056, US

(21) Appl. No.: 10/810,299

(22) Filed: Mar. 26, 2004

(30) Foreign Application Priority Data
Mar. 26, 2003 (FR) 0303176

(51) Int. Cl. A61M 16/00

(52) U.S. Cl. 128/204.18

(57) ABSTRACT

The invention relates to a portable assembly for emergency ventilation, comprising a source (1) of compressed gas equipped with a gas pressure-reducing valve device (2) with which it is possible to control the flowrate and/or the pressure of the gas issuing from the gas source (1), a respiratory assistance ventilator (3) fed with gas by said gas source (1), and a man/machine interface (4) cooperating with said ventilator (3) so as to permit regulation of at least one ventilation parameter and/or of at least one ventilation set-point. The portable assembly according to the invention constitutes an item of equipment for respiratory aid which can be used in emergency situations, is of small size and low weight, and can be easily carried by a doctor or similar, even in places where access is difficult.

The present invention relates to a portable assembly for emergency ventilation, comprising an oxygen cylinder, a mini-ventilator supplying a patient circuit which terminates in a respiration mask or similar, and a man/machine interface cooperating with the ventilator.

At present, when a doctor working for an emergency service comes to the aid of a patient in respiratory distress, he is usually equipped with a source of oxygen, for example a cylinder of compressed oxygen, and with an emergency apparatus which is connected to the gas source via a flexible tube and to the patient via a tube and a respiration mask.

However, these items of equipment are heavy and cumbersome, which means that the doctor generally needs to use both hands when transporting them.

If the location of the emergency is one where access is difficult, for example a steep site, a narrow tunnel or a cramped location, getting all the equipment there often involves several stages, that is to say going back and forth several times, or requires several persons.

Moreover, it takes some time to set up the equipment and get it ready because the doctor first has to establish all the connections, then concentrate on regulating the emergency apparatus, the flowrate and/or pressure of the gas delivered by the cylinder, the ventilation mode, for example controlled ventilation (CV) or controlled assisted ventilation (CAV), the positive expiratory pressure (PEP), the inhalation trigger threshold, the respiratory frequency, the ratio of the inhalation time to the exhalation time, the maximum safety pressure, and other ventilation or safety parameters, and must do all this before specifically dealing with the victim.

It will thus be appreciated that, in some extreme cases, the time needed for all these operations can prove fatal for the victim.

The problem thus posed in this context is that of being able to provide an apparatus for respiratory aid which can be used in emergency situations, is of small size and low weight, and can be easily transported by a doctor or similar, even in locations where access is difficult, and this requiring the use of only one of said doctor's hands, or indeed requiring no hands at all if the assembly is placed in a suitable carrier arrangement.

The solution according to the invention is therefore a portable assembly for emergency ventilation, comprising:

a source of compressed gas equipped with a gas pressure-reducing valve device with which it is possible to control the flowrate and/or the pressure of the gas issuing from the gas source,

a respiratory assistance ventilator fed with gas by said gas source, and

a man/machine interface cooperating with said ventilator so as to permit regulation of at least one ventilation parameter and/or of at least one ventilation set-point.

Depending on the circumstances, the assembly according to the invention can comprise one or more of the following technical characteristics:

the gas pressure-reducing valve device comprises an outlet connector to which the respiratory assistance ventilator is fixed;

the respiratory assistance ventilator comprises an internal gas circuit forming a fluidic connection from an inlet orifice to an outlet orifice, a proportional valve being arranged on said internal circuit, said valve being controlled by control means cooperating with the man/machine interface;

the respiratory assistance ventilator moreover comprises a venturi injector arranged on the internal circuit, downstream of the proportional valve;

the respiratory assistance ventilator additionally comprises a flowrate sensor and a pressure sensor for measuring the flowrate and the pressure of the gas in the internal circuit, said sensors cooperating with the control means in such a way as to permit automatic control and regulation of the proportional valve in terms of flowrate and/or pressure;

the man/machine interface comprises means for regulating a ventilation set-point or parameter in order to permit selection and/or regulation of at least one ventilation parameter and/or of at least one ventilation set-point, and preferably display means cooperating with said regulating means in order to make it possible to visualize and/or display at least one value of at least one ventilation parameter and/or of at least one ventilation set-point that has been selected and/or regulated;

it comprises a patient circuit with at least one gas conduit connected, via its upstream end, to the outlet orifice of the ventilator and, via its downstream end, to a respiration mask;

the pressure-reducing valve and the ventilator are protected by a protective hood fixed on the gas source;

the means for regulating a ventilation set-point or parameter permit selection and/or regulation of at least one ventilation parameter and/or of at least one ventilation set-point chosen from the group comprising the ventilation frequency, the ventilation flowrate, the ventilation volume, the composition of the gas mixture, the inhalation trigger threshold, the inhalation time and/or the exhalation time, or their ratio, the positive expiratory pressure (PEP), the ventilation mode, and the maximum safety pressure;

the pressure-reducing valve device, the respiratory assistance ventilator and the man/machine interface form a compact system supported by the gas source, in particular by an oxygen cylinder;

it has a total weight of less than 25 kg, preferably less than 15 kg, so that it can be easily carried by a doctor;

it is placed in a carrier arrangement, for example a backpack, a harness, or any similar carrying means.

The invention will be described in more detail with reference to the attached figures, which are given by way of illustration.

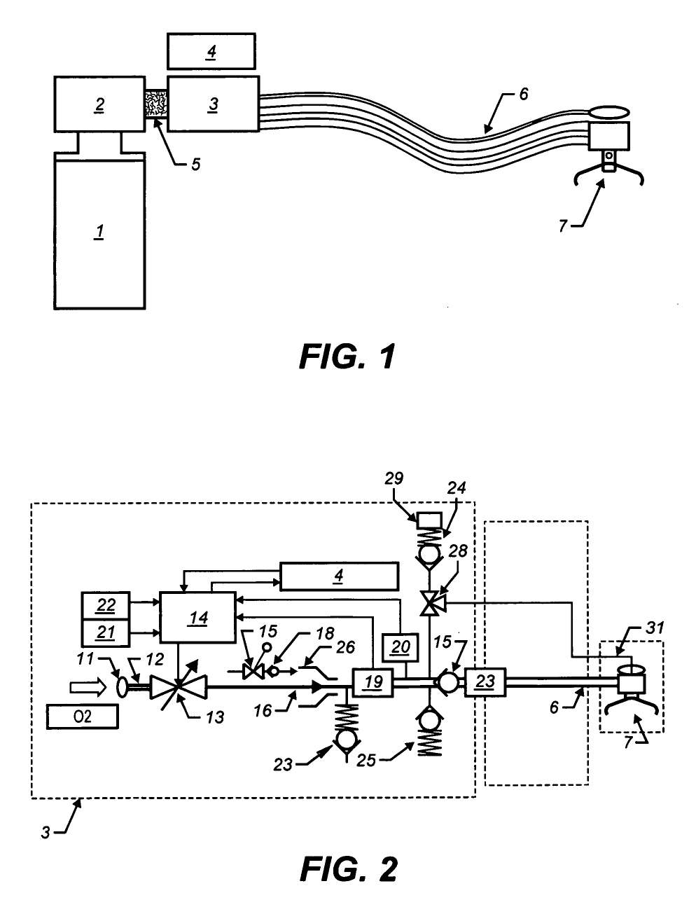

As is shown diagrammatically in FIG. 1, the assembly according to the invention is composed of three main elements, namely a gas source 1, in particular an oxygen cylinder, provided with an integrated pressure-reducing valve 2 with a standardized low-pressure outlet, and a micro-ventilator 3 which has, on its gas inlet side, a standardized low-pressure inlet connector permitting direct mounting on the outlet 5 of the pressure-reducing valve 2 belonging to the gas source 1, typically an oxygen cylinder.

The micro-ventilator 3 can for example be mounted on a gas cylinder 1 equipped with a protective hood, such as is described by documents EP-A-629812 or EP-A-811900.

The elements making up the ventilator 3, which are represented in FIG. 2, are small in size, which allows them to be accommodated within a limited volume.

Thus, in FIG. 2, it will be seen that the micro-ventilator 3 for respiratory assistance comprises a gas inlet orifice 11 which can be connected to the low-pressure outlet connector 5 of the pressure-reducing valve 2, and a gas outlet orifice 15 through which the gas is delivered to the patient circuit 6, the gas being conveyed from the inlet orifice 11 to the outlet orifice 15 via an internal gas-conveying circuit 12 on which is arranged a proportional valve 13 controlled by control means 14 comprising an electronic card. The proportional valve 13 permits regulation of the proportion of gas delivered to the patient circuit 6.

The internal circuit 12 also comprises, downstream of the proportional valve 13, an ejector unit which is able to produce a mixture of air and oxygen in volume proportions varying between 60 and 100% oxygen. This unit comprises a gas injector 16 and a straight convergent nozzle, in particular of the venturi type, connected to the outside atmosphere by way of a nonreturn valve 18 and a manually controlled mechanical distributor 17 which has at least two positions and at least two orifices, namely in particular a position corresponding to ventilation at 100% oxygen (pure 02 without addition of outside air) and a position corresponding to ventilation with an air/oxygen mixture.

The nonreturn valve 18 prevents escape of oxygen in the event of a substantial back-pressure in the patient circuit 6.

The distributor 17 permits passage of atmospheric air to the convergent/divergent nozzle 26 in order to form the air/oxygen mixture or, conversely, so as not to form this mixture if it is necessary to ventilate the patient with pure oxygen.

A flowrate sensor 19 and pressure sensor 20 are connected to the internal circuit 12, downstream of the venturi injector 16, so as to be able to measure the flowrate and pressure of the gas in the internal circuit 12 downstream of the injector 16.

By virtue of the proportional valve 13 connected to the inlet of the venturi injector 16 and to the flowrate and pressure sensors 19, 20, it is possible to control the opening and closing of said valve 13 as a function of the flowrate and pressure set-points regulated by the doctor on the man/machine interface 4 and in response to the measurements of flowrate and pressure effected by the sensors 19, 20 which cooperate with the control means 14.

Indeed, the micro-ventilator 3 also incorporates a man/machine interface 4 allowing the doctor to perform all the adjustments in a very simple manner.

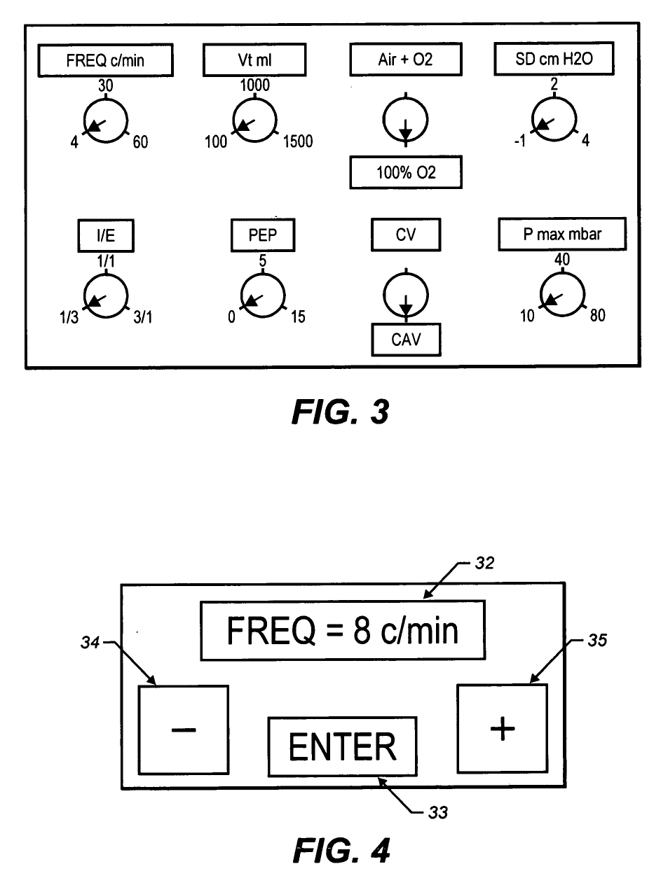

As is shown in FIG. 3, this man/machine interface 4 is composed, for example, of a frequency-regulating means (FREQ c/min), a means (Vt ml) for regulating the ventilation flowrate per minute or flow volume, a means for control of two positions (position with air+oxygen, and position with pure oxygen), a means for regulating the inhalation trigger threshold (SD cm H₂O), a means for regulating the inhalation time and the exhalation time or the ratio of the two (I/E), a means for regulating the positive expiratory pressure (PEP), a means for regulating the ventilation mode (CV/CAV), and a means for regulating the maximum safety pressure (P max mbar). These different means are, for example, rotatable knobs or any similar or equivalent actuating system.

To reduce the size of the man/machine interface 4, the system outlined in FIG. 3 can be replaced with a control system of the type in which the different ventilation parameters measured by the machine are displayed so as also to be able to inform the doctor of the correct conduct of the intervention and to further increase the safety of the patient.

Such a system is outlined in FIG. 4 where it will be seen that it comprises an information display means 32, such as an LED screen or similar, making it possible to visualize the various abovementioned parameters to be regulated (frequency, flowrate, PEP, etc.).

These parameters to be regulated are selected by way of selection means 33, 34, 35, which here comprise several selection buttons or keys which the doctor can actuate using a finger, namely + and − keys 34, 35 for selecting a parameter or function, and a key 33 for validating the parameter or function chosen.

The desired value of each parameter is also regulated with selection buttons or keys 34, 35 (+ and − keys), and the chosen value is validated by actuating the validation key 33 (key marked “ENTER”); it should be noted, however, that other specific keys can also be provided for these purposes.

As is shown in FIG. 2, the control means 14 are fed with electrical current by a current source 22, such as a rechargeable battery, cells or the like, and can also comprise a connection to the mains electricity 21.

The micro-ventilator 3 also comprises a system for regulating the PEP (positive expiratory pressure) comprising a control button 29 and a unit 24 consisting of a seat and of a membrane on which rests a spring mechanism which is compressed to a greater or lesser extent by the button 29. Said membrane to a greater or lesser extent closes the orifice connecting the patient circuit and the atmosphere by way of the solenoid valve 28 during the exhalation phase.

Moreover, the valve 25 allows the patient circuit to be connected to the atmosphere if the patient ventilation pressure reaches the critical maximum pressure in the patient circuit 6.

In other words, the PEP regulation system 24 comprises an orifice which is connected, at one end, to the circuit opening into the patient circuit 6 supplying the patient with gas and situated downstream of the assembly 26, and which, via its other end, communicates with the atmosphere when the pressure in the conduit 6 becomes greater than the adjustable pressure exerted by an elastic means 24, such as a spring, on a membrane or similar arranged at said end communicating with the atmosphere.

An exhalation solenoid 28 having three orifices and two positions is arranged between the patient circuit 6, the PEP unit 24 and the exhalation valve control 31.

In the inhalation phase, the solenoid valve 28 uses the pressure generated by the machine to pressurize the balloon of the exhalation valve so as to isolate the patient from the atmosphere and allow the fluid to feed the patient via the mask 7 or an intubation tube.

In the exhalation phase, the solenoid valve 28 isolates the exhalation valve control 31 from the patient pressure and connects it to the PEP unit 24 to allow the patient to exhale at atmospheric pressure or at the regulated positive expiratory pressure.

Similarly, the safety system 25 for the maximum pressure authorized in the internal circuit 6 comprises a conduit connected, on the one hand, to the internal circuit 6, and, on the other hand, to the atmosphere. In the event of an excessive rise in pressure in the circuit 6, the unit 25 allows the gas to pass from the circuit 6 to the outside. As before, the unit 25 is controlled by a valve on which a pressure exerted by an elastic means of the spring type is applied. The nonreturn valve 15 isolates the machine from the patient in the case where the latter forces the exhaled gas back to the machine.

The outlet of the micro-ventilator is equipped with an outlet connector, for example in the form of a 22 mm cone standardized for connection with the patient circuit 6, to which a respiration mask 7 is connected.

The assembly according to the invention can be placed in a backpack, allowing the user complete freedom of movement when accessing the place of the accident.

In other words, the solution according to the invention is based on a very compact assembly composed of an oxygen cylinder equipped with an integrated pressure-reducing valve to which an emergency ventilator of very small size is directly connected on the standardized low-pressure outlet. The valve outlet nozzle remains free for possible use of oxygen therapy nose-clips in cases of less urgency.

The emergency micro-ventilator connected directly on the socket is thus very near to the source and partly protected by the hood protecting the cylinder valve, when the cylinder is equipped with such a protective hood.

The fixation of the micro-ventilator can be reinforced by supplementary means, for example straps or other quick couplings.

The cylinder is operated with a single manoeuvre, by turning the control a ¼ of a turn.

To use the assembly, the doctor or similar has a very small number of things to do once on site, namely in particular:

opening the valve of the cylinder (a ¼ turn)

starting the ventilator by actuating an on/off switch,

regulating, for the particular patient, the respiration frequency and the ratio of the inhalation time to the exhalation time via the man/machine interface,

choosing ventilation by pure oxygen or ventilation by oxygen mixed with air with the aid of the gas injector 16, the set-point of the insufflated flowrate or the flow volume administered to the patient, and the appropriate ventilation mode,

adjusting the ventilation trigger threshold of the machine.

The micro-ventilator 3 is thus supplied directly via the outlet orifice of the cylinder 1 to which it is firmly connected, the outlet of the micro-ventilator 3 being in turn connected directly to the patient circuit 6, at the end of which is the respiration mask 7 with an exhalation valve 31.

1-10 (cancelled).

11. A portable assembly apparatus for emergency ventilation, comprising:
a) a source of compressed gas, wherein said compressed gas course is equipped with a gas pressure-reducing valve device to control the flowrate and the pressure of the gas issuing from said compressed gas source;
by a respiratory assistance ventilator fed with gas by said compressed gas source; and
c) a man/machine interface cooperating with said ventilator so as to permit regulation of at least one ventilation parameter and at least one ventilation set-point.

12. The apparatus according to claim 11, wherein said gas pressure-reducing valve device comprises an outlet connector to which said respiratory assistance ventilator is fixed.

13. The apparatus according to claim 11, wherein said respiratory assistance ventilator comprises an internal gas circuit forming a fluidic connection from an inlet orifice to an outlet orifice, and a proportional valve being arranged on said internal circuit, said valve being controlled by control means cooperating with said man/machine interface.

14. The apparatus according to claim 13, wherein said respiratory assistance ventilator further comprises a venturi injector arranged on said internal circuit, downstream of said proportional valve.

15. The apparatus according to claim 13, wherein said respiratory assistance ventilator further comprises a flowrate sensor and a pressure sensor for measuring the flowrate and the pressure of the gas in the internal circuit, said sensors cooperating with said control means in such a way as to permit automatic control and regulation of said proportional valve in terms of flowrate or pressure.

16. The apparatus according to claim 11, wherein said man/machine interface comprises means for regulating a ventilation set-point or parameter in order to permit selection and regulation of at least one ventilation parameter or of at least one ventilation set-point.

17. The apparatus according to claim 16, further comprising display means cooperating with said regulating means in order to make it possible to visualize and display at least one value of at least one ventilation parameter or of at least one ventilation set-point that has been selected and regulated.

18. The apparatus according to claim 13, wherein further comprising a patient circuit with at least one gas conduit connected, via its upstream end, to said outlet orifice of said ventilator and, via its downstream end, to a respiration mask.

19. The apparatus according to claim 11, wherein said pressure-reducing valve and said ventilator are protected by a protective hood fixed on said compressed gas source.

20. The apparatus according to claim 16, wherein said means for regulating a ventilation set-point or parameter permit selection and regulation of at least one ventilation parameter or of at least one ventilation set-point are selected from the group consisting of:
a) ventilation frequency;
b) ventilation flowrate;
c) ventilation volume;
d) composition of the gas mixture;
e) inhalation trigger threshold;
f) inhalation time;
g) exhalation time
h) inhalation time and exhalation time;
i) ratio of inhalation time and exhalation time;
j) positive expiratory pressure (PEP);
k) ventilation mode; and
l) maximum safety pressure.

21. The apparatus according to claim 11, wherein said pressure-reducing valve device, said respiratory assistance ventilator, and said man/machine interface cooperating with said ventilator form a compact system supported by said compressed gas source.

22. The apparatus according to claim 11, wherein said compact system is supported by an oxygen cylinder.

23. The apparatus according to claim 11, wherein the total weight is less than 25 kg.

24. The apparatus according to claim 23, wherein the total weight is less than 15 kg.

25. The apparatus according to claim 11, further comprising a carrier arrangement.

26. The apparatus according to claim 25, wherein said carrier arrangement is selected from the group consisting of:
a) backpack;
b) harness; and
c) any similar carrying means.

27. A portable assembly apparatus for emergency ventilation, comprising:
a) a source of compressed gas, wherein said compressed gas course is equipped with a gas pressure-reducing valve device to control the flowrate and the pressure of the gas issuing from said compressed gas source;
b) a respiratory assistance ventilator fed with gas by said compressed gas source; and
c) a man/machine interface cooperating with said ventilator so as to permit regulation of at least one ventilation parameter and at least one ventilation set-point,
wherein said gas pressure-reducing valve device comprises an outlet connector to which said respiratory assistance ventilator is fixed;
wherein said respiratory assistance ventilator comprises an internal gas circuit forming a fluidic connection from an inlet orifice to an outlet orifice, and a proportional valve being arranged on said internal circuit, said valve being controlled by control means cooperating with said man/machine interface;
wherein said man/machine interface comprises means for regulating a ventilation set-point or parameter in order to permit selection and regulation of at least one ventilation parameter or of at least one ventilation set-point;
wherein said pressure-reducing valve and said ventilator are protected by a protective hood fixed on said compressed gas source;
wherein said pressure-reducing valve device, said respiratory assistance ventilator, and said man/machine interface cooperating with said ventilator form a compact system supported by said compressed gas source; and
wherein said compact system is supported by an oxygen cylinder.

28. The apparatus according to claim 27, wherein the total weight is less than 15 kg, and further comprises a carrier arrangement selected from the group consisting of:
a) backpack;
b) harness; and
c) any similar carrying means.

29. A method of providing emergency ventilation to a patient comprising treating said patient with oxygen, wherein said oxygen is provided using a portable assembly comprising:
a) a source of compressed gas, wherein said compressed gas course is equipped with a gas pressure-reducing valve device to control the flowrate and the pressure of the gas issuing from said compressed gas source;
b) a respiratory assistance ventilator fed with gas by said compressed gas source; and
c) a man/machine interface cooperating with said ventilator so as to permit regulation of at least one ventilation parameter and at least one ventilation set-point.
